SEQUENCE LISTING (26 sequences)

SEQ ID NO: 1 - DNA, 4520 bp, Homo sapiens
taggaacaggggagagtgcacctgctaccagtcaagctcagccagactgcaagaggaggcgaggcggagccagccgagggagtgaaccatggacaagttgaaatgcccgagtttcttcaagtgcagggagaaggagaaagtgtcggcttcatcagagaatttccatgttggtgaaaatgatgagaatcaggaccgtggtaactggtccaaaaaatcggattatcttctatctatgattggatacgcagtgggattaggaaatgtgtggagatttccatatctgacctacagcaatggtggaggcgccttcttgataccttatgcaattatgttagcattggctggtttacctttgttctttctggagtgttcactgggacaatttgctagcttaggtccagtttcagtttggaggattcttccattgtttcaaggtgtgggaattacaatggtcctgatctccatttttgtgacaatctattacaatgtcataattgcctatagtctttactacatgtttgcttcttttcaaagtgaactaccatggaaaaattgttcttcgtggtcagataaaaactgtagcagatcaccaatagtaactcactgtaatgtgagtacagtgaataaaggaatacaagagatcatccaaatgaataaaagctgggtagacatcaacaattttacctgcatcaacggcagtgaaatttatcagccagggcagcttcccagtgaacaatattggaataaagtggcgctccaacggtcaagtggaatgaatgagactggagtaattgtttggtatttagcactttgtcttcttctggcttggctcatagttggagcagcactatttaaaggaatcaaatcgtctggcaaggtggtatattttacagctcttttcccctatgtggtcctactcatcctgttagtacgaggtgcaactctggagggtgcttcaaaaggcatttcatactatattggagcccagtcaaattttacaaaacttaaggaagctgaggtatggaaagatgctgccactcagatattttactccctttcagtggcttggggtggcttagttgctctatcatcttacaataagttcaaaaacaactgcttctctgatgccattgtggtttgtttgacaaactgtctcactagcgtgtttgctggatttgctattttttctatattgggacacatggcccatatatctggaaaggaagtttctcaagttgtaaaatcaggttttgatttggcattcattgcctatccagaggctctagcccaactcccaggtggtccattttggtccatattattttttttcatgcttttaactttgggtctcgattctcagtttgcttcgattgaaacgatcacaacaacaattcaagatttatttcccaaagtgatgaagaaaatgagggttcccataactttgggctgctgcttggttttgtttctccttggtctcgtctgtgtgactcaggctggaatttactgggttcatctgattgaccacttctgtgctggatggggcattttaattgcagctatactggagctagttggaatcatctggatttatggagggaacagattcattgaggatacagaaatgatgattggagcaaagaggtggatattctggctatggtggagagcttgctggtttgtaattacgcctatccttttgattgcaatatttatctggtcattggtgcaatttcatagacctaattatggcgcaattccataccctgactggggagttgctttaggctggtgtatgattgttttctgcattatttggataccaattatggctatcataaaaataattcaggctaaaggaaacatctttcaacgccttataagttgctgcagaccagcttctaactggggtccatacctggaacaacatcgtggggaaagatataaagacatggtagatcctaaaaaagaggctgaccatgaaatacctactgttagtggcagcagaaaaccggaatgagatctcattgaaaaaaatatatgattgtataatgtgattttttttagaatagggggaaccttatttatttgtgtgttaactgaataggaaaatgtacatactatgttcatgatagtgtgatttttttcacatttaagcaggaatgcaatataaaaatgtgaatctcttaattctcagccatgtgcttattatatttctttttagattgtctatctgtataacacacacacacacacctaagagtctctatttcacaattatatttttgtaaatagtatatgcatttttaatacattggaggctttattttgaactaatttcttagagaatagttatattttctattacacaagtttaaaaatattattaacttgtattttcttaatatacaatctatcttttccacaaatatgagtgggaaataaatcagcacatttgaaagaaagtgttaaaactgaaggcctcacttaattagaaacgtgataaatatatggacaaatggactatacatactataagaggactgtagtttaatactttttacccaaatatgtttaaaaacatcgtgcatttgttacagctcatgttttctatatgaacttagtcattaatgttctttataaaaagtgaaataagatggaaaaattaggatcctacagccagtacgtgataaatctagaaaattgagttttgagtacctcttttcccatatacaatcttccttccttaggtaatttggaagaaaactatgacccatttaatttctattgtgtttcaccaaattcagtgttgttcattatacctctctgaaatataggtttaatttcaaatagaatatggacttaaatgttaatgagaaactggctttaatcaattctagcattttattactgtaatacagggctgatagagtgattttgtcttatatgagtcagttactacttacaggtgataacttgcatactattggaagataaagttgtcaaacttgtcaagaatgagaaaagccaaattagaaaatcctatgtcctagtttccttaccaaggataattaaatatatcactaagagctttatatattgattatatattgttgacaactggtttaagcatcatagcctatgatgataaacactgcctatatatgtaaatagcttttcatcaattcttaaatttcttaacctaggcttcagggagcatatgaaaccaaaattatatggaacattttctgtgtgtacatgtacatgcatttttctagggagagagtccgtaggtttatcagaatatcaaggaaaactgtgacccaaagaagtttaagaatcacatacagtgctgctggctttttgtgcttggcaaatgagtgacaatagaagaaataatttttcttacacattttaaaacgttttctcttccttgtgattgaagatgaaaggagtaagaaattaaggcatttgtttaatttatactggtaacttatttaggggggaggggacatgaaggtaggtaaataggtaggcctctaattgaaccacctctctaagttatgtacgtatatataagctgaaattgtgtttgacattctgagggttttctttttctttttcctttttttttttttttggtggggggctgggggtcagagtcttgttctgttgcctgggctggagtgcagtggcatgatctcagctcactgcaacctctgccttctggattcaagtgattctcctgcctcagcctcttgagtagctgggactacaggtgcccgccaccacaccagctaatttttgtatttttagtagaggcgaagtttccccatgttggccaggctggtcttgaactcccgacctcaagtgatctgtctacctcggcctcctaaagtgctgagattacaggtgtgagccaccgtgcccggcccattctaagggttttctttgaagacaggtcaaatgctgttagtaagtttcaggagattgttaattcctcagttataccagattttataaaatatttgagaatagatggctaacaagaggttagaaatacttttccttaattttaatccacagtatgttacatgcattctaccactacattttggtgctatttaaggtgtgcaattttctataggtgacttttgcaattcagggaagatttgggcatattaaatgaaagaatatctaattgggggaggtgtgaagggaaagaaattcttttcaaaagctgaccacaaagagtagttaaaagtttttgtcactatcttcacaagtgtgtaaagcacagatttcaacagagtgcttggcatattgtagggtgctcaatggtggtttttattattattactcagattccacagtggcaagaaacatcattctacataatggaaaacatttacatcaaatcccacttactttaatgcgaacttggagataatttatggtattgtattgtaaaccattaatgaaaactttttcacagttgagtgaaattaaaatcactatatctc

SEQ ID NO: 2 - DNA, 2832 bp, Homo sapiens
aggaagctgaaccatctatctccagaaatgtcttcagaaagtaaagagcaacataacgtttcacccagagactcagctgaaggaaatgacagttatccatctgggatccatctggaacttcaaagggaatcaagtactgacttcaagcaatttgagaccaatgatcaatgcagaccttatcataggatccttattgagcgtcaagagaaatcagatacaaacttcaaggagtttgttattaaaaagctgcagaagaattgccagtgcagtccagccaaagccaaaaatatgattttaggtttccttcctgttttgcagtggctcccaaaatacgacctaaagaaaaacattttaggggatgtgatgtcaggcttgattgtgggcatattattggtgccccagtccattgcttattccctgctggctggccaagaacctgtctatggtctgtacacatctttttttgccagcatcatttattttctcttgggtacctcccgtcacatctctgtgggcatttttggagtactgtgccttatgattggtgagacagttgaccgagaactacagaaagctggctatgacaatgcccatagtgctccttccttaggaatggtttcaaatgggagcacattattaaatcatacatcagacaggatatgtgacaaaagttgctatgcaattatggttggcagcactgtaacctttatagctggagtttatcaggtagcgatgggcttctttcaagtgggttttgtttctgtctacctctcagatgccttgctgagtggatttgtcactggtgcctccttcactattcttacatctcaggccaagtatcttcttgggctcaaccttcctcggactaatggtgtgggctcactcatcactacctggatacatgtcttcagaaacatccataagaccaatctctgtgatcttatcaccagccttttgtgccttttggttcttttgccaaccaaagaactcaatgaacacttcaaatccaagcttaaggcaccgattcctattgaacttgttgttgttgtagcagccacattagcctctcattttggaaaactacatgaaaattataattctagtattgctggacatattcccactgggtttatgccacccaaagtaccagaatggaacctaattcctagtgtggctgtagatgcaatagctatttccatcattggttttgctatcactgtatcactttctgagatgtttgccaagaaacatggttacacagtcaaagcaaaccaggaaatgtatgccattggcttttgtaatatcatcccttccttcttccactgttttactactagtgcagctcttgcaaagacattggttaaagaatcaacaggctgccatactcagctttctggtgtggtaacagccctggttcttttgttggtcctcctagtaatagctcctttgttctattcccttcaaaaaagtgtccttggtgtgatcacaattgtaaatctacggggagcccttcgtaaatttagggatcttcccaaaatgtggagtattagtagaatggatacagttatctggtttgttactatgctgtcctctgcactgctaagtactgaaataggcctacttgttggggtttgtttttctatattttgtgtcatcctccgcactcagaagccaaagagttcactgcttggcttggtggaagagtctgaggtctttgaatctgtgtctgcttacaagaaccttcagactaagccaggcatcaagattttccgctttgtagcccctctctactacataaacaaagaatgctttaaatctgctttatacaaacaaactgtcaacccaatcttaataaaggtggcttggaagaaggcagcaaagagaaagatcaaagaaaaagtagtgactcttggtggaatccaggatgaaatgtcagtgcaactttcccatgatcccttggagctgcatactatagtgattgactgcagtgcaattcaatttttagatacagcagggatccacacactgaaagaagttcgcagagattatgaagccattggaatccaggttctgctggctcagtgcaatcccactgtgagggattccctaaccaacggagaatattgcaaaaaggaagaagaaaaccttctcttctatagtgtgtatgaagcgatggcttttgcagaagtatctaaaaatcagaaaggagtatgtgttcccaatggtctgagtcttagtagtgattaattgagaaggtagatagaagaatgtctagccaataggttaaaatttcaagtgtccaacatttcccagttccacagtgggaaattttgcacacttgaaattttaaccaagtggctagatattattcctcctttgaagctaatggcatttgtatatacacactgcagcagagcttgtagctggacagagtcaaaaagaagaaaatacggtttcaggctttcttgcagatatgaagtattcttggaatgcaataagtatgtattgaactgtactgtaaagtagctccaaaacttaattactctcctgttttaggggttatacatttggactgtgcattctccaagagatgaagcggtgaagttgggatttacattggaagtgctgtagacttctttatgtggctcagtggagagagggaaagaatgttgcacctgctctagtaccataggtcaagaggcttctggatcacaaagtcataactagacaggtttgttcttgtagttttctatccccagtctttgctccccagatggcagtagtttttagtaggaaagtgccattcctgtccttaaggcacagtctcatcag

SEQ ID NO: 3 - DNA, 1103 bp, Homo sapiens
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatag

SEQ ID NO: 4 - DNA, 1140 bp, Homo sapiens
ggcacgagggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccacgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcttccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 5 - DNA, 345 bp, Homo sapiens
atgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaa

SEQ ID NO: 6 - DNA, 1180 bp, Homo sapiens
cactgaagaaggcaggggcccttagagtcttggttgccaaacagatttgcagatcaaggagaacccaggagtttcaaagaagcgctagtaaggtctctgagatccttgcactagctacatcctcagggtaggaggaagatggcttccagaagcatgcggctgctcctattgctgagctgcctggccaaaacaggagtcctgggtgatatcatcatgagacccagctgtgctcctggatggttttaccacaagtccaattgctatggttacttcaggaagctgaggaactggtctgatgccgagctcgagtgtcagtcttacggaaacggagcccacctggcatctatcctgagtttaaaggaagccagcaccatagcagagtacataagtggctatcagagaagccagccgatatggattggcctgcacgacccacagaagaggcagcagtggcagtggattgatggggccatgtatctgtacagatcctggtctggcaagtccatgggtgggaacaagcactgtgctgagatgagctccaataacaactttttaacttggagcagcaacgaatgcaacaagcgccaacacttcctgtgcaagtaccgaccatagagcaagaatcaagattctgctaactcctgcacagccccgtcctcttcctttctgctagcctggctaaatctgctcattatttcagaggggaaacctagcaaactaagagtgataagggccctactacactggcttttttaggcttagagacagaaactttagcattggcccagtagtggcttctagctctaaatgtttgccccgccatccctttccacagtatccttcttccctcctcccctgtctctggctgtctcgagcagtctagaagagtgcatctccagcctatgaaacagctgggtctttggccataagaagtaaagatttgaagacagaaggaagaaactcaggagtaagcttctagcccccttcagcttctacacccttctgccctctctccattgcctgcaccccaccccagccactcaactcctgcttgtttttcctttggccatgggaaggtttaccagtagaatccttgctaggttgatgtgggccatacattcctttaataaaccattgtgtacataaaaaaaaaaaaaaaaaaaaaaaaaaaaaaaaaaaa

SEQ ID NO: 7 - DNA, 3109 bp, Homo sapiens
cattggacttcagcatgactactcagttgccagcttacgtggcaattttgcttttctatgtctcaagagccagctgccaggacactttcattgcagctgtttatgagcatgcagcgatattgcccaatgccaccctaacaccagtgtctcgtgaggaggctttggcattaatgaatcggaatctggacattttggaaggagcgatcacatcagcagcagatcagggtgcgcatattattgtgactccagaagatgctatttatggctggaacttcaacagggactctctctacccatatttggaggacatcccagaccctgaagtaaactggatcccctgtaataatcgtaacagatttggccagaccccagtacaagaaagactcagctgcctggccaagaacaactctatctatgttgtggcaaatattggggacaagaagccatgcgataccagtgatcctcagtgtccccctgatggccgttaccaatacaacactgatgtggtatttgattctcaaggaaaactggtggcacgctaccataagcaaaaccttttcatgggtgaaaatcaattcaatgtacccaaggagcctgagattgtgactttcaataccacctttggaagttttggcattttcacatgctttgatatactcttccatgatcctgctgttaccttggtgaaagatttccacgtggacaccatagtattcccaacagcttggatgaatgttttgccacatttgtcagctgttgaattccactcagcttgggctatgggcatgagggtcaatttccttgcatccaacatacattacccctcaaagaaaatgacaggaagtggcatctatgcacccaattcttcaagagcatttcattatgatatgaagacagaagagggaaaactcctcctctcgcaactggattcccacccatcccattctgcagtggtgaactggacttcctatgccagcagtatagaagcgctctcatcaggaaacaaggaatttaaaggcactgtctttttcgatgaattcacttttgtgaagctcacaggagttgcaggaaattatacagtttgtcagaaagatctctgctgtcatttaagctacaaaatgtctgagaacataccaaatgaagtgtacgctctaggggcatttgacggactgcacactgtggaagggcgctattatctacagatttgtaccctgttgaaatgtaaaacgactaatttaaacacttgcggtgactcagctgaaacagcttctaccaggtttgaaatgttctccctcagtggcactttcggaacccagtatgtctttcctgaggtgttgctgagtgaaaatcagcttgcacctggagaatttcaggtgtcaactgacggacgcttgtttagtctgaagccaacatccggacctgtcttaacagtaactctgtttgggaggttgtatgagaaggactgggcatcaaatgcttcatcaggcctcacagcacaagcaagaataataatgctaatagttatagcacctattgtatgctcattaagttggtagaatattgactttttctcttttttatttgggataatttaaaaaatgatggatgagaaaagaaagattggtccgggttaatattatcctctagtataagtgaattactagtttctctttatttagacaaacacacacacaccagataatataaacttaataaattatctgttaatgtagattttatttaaaaaactatatttgaacattggtctttcttggacgtgagctaattatatcaaataagtatcacaaatcttttacgcagaagaaataaaaactacgggtagaaaacataagaactatcataaaatttacttacaaggaggctgctcttgttaccacttttattatattacgtatcacttattcagctctgctgaaaatttccaatgactttgtttgtttgctcttttagttttttacctaaacaatacattttgattctcttgtgggttgataatgtctccccaaaatttacatgttgaagcacctcagaatgtgactgtatttggagacagggtctttaaagaggtaaaataaggtcattaggatagaccctaattcaatatgactgatgatcataaaagaagaggcgagtagggcacaacaggcacaaagggagaccataaggagacacagaggaaggacaactctttacaagctaagaagagagggcctcagaagaaaccaaccctgccaacaccttgatcttggacttccagcctccaaaactatgagaaataaatttctattgtttaagtcacccagtccatggtactttgttaggcagccctggcaaatgaatcaaagacccattcctgttcctctccccaccactactgttttctactgtaatctgaagcttcaacaaaaggcttacctggtaagaatattcagctggtctgggtcctcaagactccaatagacactcttaaagaaggattgctgatggattgatagtgaaaccattagatcattgaattcctctggaattagaaaaccagagagtcccattttaagaaattagatatttaatatagcattgtgtgttctattttagtaacagcagaatctcttgacattacacaactcagtgaaacaacatcatttaagccaaaatatctcccaactgactgatagactctgagcactaatatcatagtgctgtgatgatggacaattacatagtaccgataacagccatgcactgtgcaaagcatgcccttctgcacaggagagcaaggcacttgcagtagtgatctatgccagcaaaacatcattttgagacaaacatttttgtggcagatgtttttcctaaaaagtactatatcatccaagaaatatttgagtaaaatcccttgttcttttgggtgacattaactgacatttgctttttttcaagacctaatagaaaataagaaagcccataatgtatttagaaacaggaatcctcagagcaattctctgtattctcatataatttcaatgtaaaacagaaaacatattgatgtgttggtgataggcttgaattattaaaaacttcaaaaacaaaa

SEQ ID NO: 8 - DNA, 60 bp, Artificial Sequence
  Description of Artificial Sequence Synthetic oligonucleotide
tagtctatgatcgtcgacggctgatgaagcggccgctggagttttttttttttttttttv

SEQ ID NO: 9 - DNA, 18 bp, Artificial Sequence
  Description of Artificial Sequence Synthetic oligonucleotide
tgatgaagcggccgctgg

SEQ ID NO: 10 - DNA, 20 bp, Artificial Sequence
  Description of Artificial Sequence Synthetic oligonucleotide
ttcatcagccgtcgacgatc

SEQ ID NO: 11 - DNA, 23 bp, Artificial Sequence
  Description of Artificial Sequence Synthetic oligonucleotide
cgtaagcttggatcctctagagc

SEQ ID NO: 12 - DNA, 23 bp, Artificial Sequence
  Description of Artificial Sequence Synthetic oligonucleotide
tgcaggtaccggtccggaattcc

SEQ ID NO: 13 - DNA, 24 bp, Artificial Sequence
  Description of Artificial Sequence Synthetic primer
gtgctgagattacaggtgtgagcc

SEQ ID NO: 14 - DNA, 24 bp, Artificial Sequence
  Description of Artificial Sequence Synthetic primer
cccttcacacctcccccaattaga

SEQ ID NO: 15 - DNA, 24 bp, Artificial Sequence
  Description of Artificial Sequence Synthetic primer
gtggagagagggaaagaatgttgc

SEQ ID NO: 16 - DNA, 24 bp, Artificial Sequence
  Description of Artificial Sequence Synthetic primer
ccagtttaggacagattccatggg

SEQ ID NO: 17 - DNA, 24 bp, Artificial Sequence
  Description of Artificial Sequence Synthetic primer
gtgcctaatgtgtttgagcatcgc

SEQ ID NO: 18 - DNA, 24 bp, Artificial Sequence
  Description of Artificial Sequence Synthetic primer
gcccctttgttctaagccagaaac

SEQ ID NO: 19 - DNA, 26 bp, Artificial Sequence
  Description of Artificial Sequence Synthetic primer
caggcagaacatccattcattcattc

SEQ ID NO: 20 - DNA, 24 bp, Artificial Sequence
  Description of Artificial Sequence Synthetic primer
gacatctacccactgcaagtatag

SEQ ID NO: 21 - DNA, 24 bp, Artificial Sequence
  Description of Artificial Sequence Synthetic primer
ccgtcggaaacaaggcagatggag

SEQ ID NO: 22 - DNA, 24 bp, Artificial Sequence
  Description of Artificial Sequence Synthetic primer
gaaggacgtgtgagcaggatggga

SEQ ID NO: 23 - DNA, 24 bp, Artificial Sequence
  Description of Artificial Sequence Synthetic primer
ggtgggaacaagcactgtgctgag

SEQ ID NO: 24 - DNA, 24 bp, Artificial Sequence
  Description of Artificial Sequence Synthetic primer
ggctggagatgcactcttctagac

SEQ ID NO: 25 - DNA, 24 bp, Artificial Sequence
  Description of Artificial Sequence Synthetic primer
gccagcaaaacatcattttgagac

SEQ ID NO: 26 - DNA, 24 bp, Artificial Sequence
  Description of Artificial Sequence Synthetic primer
gcctatcaccaacacatcaatatg

(19) United States
(12) Patent Application Publication
(10) Pub. No.: US 2008/0193945 A1
(43) Pub. Date: Aug. 14, 2008

(54) Method and Kit for the Diagnosis of Ulcerative Colitis

(75) Inventors: Andreas Dieckmann, Bromma (SE); Robert Lofberg, Djursholm (SE); Oliver Von Stein, Spanga (SE); Petra Von Stein, Spanga (SE)

Correspondence Address: Lisa A. Haile, J.D., Ph.D.;DLA PIPER US LLP, Suite 1100, 4365 Executive Drive, San Diego, CA, 92121-2133, US

(73) Assignee: INDEX DIAGNOSTICS AB

(21) Appl. No.: 12/100,299

(22) Filed: Apr. 9, 2008

Related U.S. Application Data
(63) Continuation of application No. 10/469,587, filed on Jul. 16, 2004, now Pat. No. 7,378,239.
(60) Provisional application No. 60/409,213, filed on Sep. 10, 2002.
(60) Provisional application No. 60/407,713, filed on Sep. 4, 2002.
(60) Provisional application No. 60/395,631, filed on Jul. 15, 2002.
(60) Provisional application No. 60/395,629, filed on Jul. 15, 2002.

(30) Foreign Application Priority Data
Jun. 25, 2002 (SE) 0201954-5
Jun. 25, 2002 (SE) 0201956-0
Jul. 18, 2002 (SE) 0202251-5
Jul. 18, 2002 (SE) 0202252.3
Jul. 18, 2002 (SE) 0202256-4

(51) Int. Cl. C12Q 1/68

(52) U.S. Cl. 435/6

(57) ABSTRACT

The differentiation between ulcerative colitis and Crohn's disease is made possible by a multi-gene approach where gene expression profiles in biopsy samples obtained from inflamed, and optionally also non-inflamed, areas in the intestines of a patient are studied.

### CROSS REFERENCE TO RELATED APPLICATIONS

This application is a continuation application of U.S. application Ser. No. 10/469,587 filed Jul. 16, 2004, now pending; which is a 35 USC §371 National Stage application of PCT Application No. PCT/SE2003/001105 filed Jun. 25, 2003; which claims the benefit under 35 USC §119(e) to U.S. Application Ser. No. 60/409,213 filed Sep. 10, 2002, now abandoned; U.S. Application Ser. No. 60/407,713 filed Sep. 4, 2002, now abandoned; U.S. Application Ser. No. 60/407,682 filed Sep. 4, 2002, now abandoned; U.S. Application Ser. No. 60/395,631 filed Jul. 15, 2002, now abandoned and U.S. Application Ser. No. 60/395,629 filed Jul. 15, 2002, now abandoned. PCT Application No. PCT/SE2003/001105 filed Jun. 25, 2003 also claims the benefit under 35 USC §119(a) to Sweden Application No. 0201954-5 filed Jun. 25, 2002; Sweden Application No. 0201956-0 filed Jun. 25, 2002; Sweden Application No. 0202251-5 filed Jul. 18, 2002; Sweden Application No. 0202252-3 filed Jul. 18, 2002 and Sweden Application No. 0202256-4 filed Jul. 18, 2002. The disclosure of each of the prior applications is considered part of and is incorporated by reference in the disclosure of this application.

### BACKGROUND OF THE INVENTION

1. Field of the Invention

The present invention relates the diagnosis of inflammatory bowel diseases, and in particular to a method and kit for the prediction and/or diagnosis of ulcerative colitis.

The invention discloses specific marker genes whose change in expression status, either collectively or a sub-set thereof, is indicative of ulcerative colitis. The present invention further relates to DNA-related methods by which quantification of the expression levels of said disease-associated marker genes directly from a biopsy allows for an immediate and accurate diagnostic test for disease type, and/or the assessment of the effect of a particular treatment regimen. The invention further discloses diagnostic kits for the detection of the expressions levels of said genes.

2. Background Information

Inflammatory bowel disease (IBD) is a term encompassing several conditions involving chronic inflammation in the gastrointestinal tract. Two of the most debilitating forms of IBD are ulcerative colitis (UC) and Crohn's disease (CD). These diseases affect young people, with a typical debut at the age of 20-30 years, and disease management is a long-term commitment for both patient and physician, since there is currently no cure for either condition. Approximately 30% of IBD patients undergo surgery during their lifetime and patients with long-standing IBD are at considerable risk of developing colorectal cancer. Three out of ten IBD patients do not respond to the best available medical therapy today, even when high doses are used, causing considerable side effects.

Treatment of patients with active UC aims at reducing inflammation and promoting colon healing and mucosal recovery. The underlying cause of UC is not understood, nor is it known what triggers the disease to recur between its inactive and active forms. However, the active stage of the disease is characterised by significant inflammation of the mucosa, increased cell permeability, loss of protein and fluids. In severe stages deep inflammation of the bowel wall may develop with abdominal tenderness, tachycardia, fever and risk of bowel perforation.

One early symptom of ulcerative colitis is constipation with passage of blood or mucus in the stools. Several months or years may pass before diarrhoea develops with abdominal pain. Later symptoms include severe fatigue, weight loss, loss of appetite, fever and occasionally arthralgia.

The road to an established diagnosis of ulcerative colitis often includes a thorough study of the patient's medical history, the exclusion of other conditions, as well as several tests, e.g. blood tests, stool examination, barium enema X-ray, sigmoidoscopy, colonoscopy, and biopsy. The biopsy may be performed as part of a sigmoidoscopy or colonoscopy examination.

It is obvious that a possibility to clinically distinguish UC from colonic CD at an early stage would provide enormous benefits for both the patient and the physician. It would permit the design of accurate treatment regimes, prevent unnecessary medications and reduce treatment costs. Even though the overall clinical picture in IBD patients may show some clinically important differences between the major patient groups of UC and CD, there are substantial similarities, thus making it difficult for health care personnel to establish a correct diagnosis.

### PRIOR ART

The prior art indicates that the available methods for distinguishing between forms of IBD, and in particular the differentiation between UC and CD, apart from the above given examples of different examination procedures, have been focused on antibody based methods.

For example WO 03/036262 describes a method and apparatus for the differentiation of Crohn's disease from other gastrointestinal illnesses, such as ulcerative colitis and irritable bowel syndrome, using the presence of faecal anti-Saccharomyces cerevisiae antibodies (ASCA) as a marker for Crohn's disease are provided. The apparatus includes an enzyme-linked immunoassay or other immunoassay that utilizes antibodies specific to human immunoglobins for the measurement of total endogenous ASCA in a human faecal sample. The method and apparatus may be used by healthcare providers to distinguish Crohn's disease from other gastrointestinal illnesses, such as ulcerative colitis and irritable bowel syndrome.

WO 01/58927 describes diagnostic methods for detecting diseases associated with an autoantigen response to hTM in affected tissue, and in particular ulcerative colitis.

There remains a need for improved methods for the accurate, rapid and reliable diagnosis of ulcerative colitis, in particular in the context of distinguishing between ulcerative colitis and Crohn's disease in IBD patients.

One aim of the present invention is to make available such methods and kits for this purpose. One particular aim is to make available a method and kit which makes it possible to reach a reliable diagnosis at an early stage of the disease. Another aim is to make it possible to distinguish between CD and UC also in difficult cases, where the clinical picture may be very similar.

Further aims underlying the invention, as well as the solutions offered by the invention and the associated advantages will become evident to a skilled person upon study of the description, examples and claims.

### SUMMARY OF THE INVENTION

The present inventors have surprisingly found that the differentiation between ulcerative colitis and Crohn's disease is made possible by a multi-gene approach where the gene expression profiles in biopsy samples obtained from inflamed and optionally also non-inflamed areas in the intestines of a patient are studied.

The present invention is based on the discovery of potential marker genes which, either collectively or in sub-groups, are indicative of the human condition of ulcerative colitis (UC). The present inventors surprisingly found that quantification of the expression levels of a number of specific genes can be utilized in accurately and simply diagnosing from a biopsy, whether the patient is afflicted with the condition of UC or, for example, Crohn's disease.

More specifically, methods are provided that allow nucleic acid amplification of seven (7) distinct genetic markers or sub-groups thereof, using pre-selected gene specific primers that allow for semi quantification of the expression levels of said genetic markers. The gene specific primers are designed to hybridise to opposing strands of the DNA encoding the genetic marker of interest such that though PCR amplification, a defined region of the encoding DNA of the genetic marker gene is produced. An assay and kit for the detection and monitoring expression status of said seven marker genes or sub-sets thereof in a biological sample are provided. The assay is a non-culture, PCR-based assay for the detection of said marker genes.

### BRIEF DESCRIPTION OF THE DRAWINGS

The present invention will be described in closer detail in the following description and examples, with reference to the drawings in which

FIG. 1 shows the RT-PCR analysis of expression status of seven marker genes on biopsy samples from patients afflicted with either ulcerative colitis (UC) or with Crohn's Disease (CD). The experimental protocol is out-lined in Example 6. (Key: M, is a base-pair marker, H, represents a biopsy from a totally normal healthy individual, I, represents a biopsy sample taken from an inflamed area and N, represents a biopsy taken from a non-inflamed area from the same patient. Numbers at the bottom of the figure indicates patient number and the vertical black lines indicate an N and I biopsy sample derived from the same patient). Gamma actin was used as a loading control and indicates the expression status of a house-keeping gene used commonly to demonstrate equal mRNA input in all RT-PCR reactions.

### DETAILED DESCRIPTION OF THE INVENTION

Before the present invention is disclosed and described, it is to be understood that one skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations of the scope of the invention. Changes therein and other uses that will occur to those skilled in the art are encompassed within the spirit of the invention as defined by the scope of the claims.

As used herein, the term “complementary DNA primer” means an oligonucleotide, which anneals to the RNA template in a particular orientation to allow for the synthesis of a nascent DNA strand in the presence of reverse transcriptase in the biological sample under the conditions described herein.

Also as used herein, the “condition” under which a DNA strand is synthesized include the presence of nucleotides, cations and appropriate buffering agents in amounts and at temperatures, such that the RNA template and the DNA primer will anneal and oligonucleotides will be incorporated into a synthesized DNA strand if reverse transcriptase is not inhibited by the reverse transcriptase inhibitor drug. Exemplary conditions are set forth in the examples below. The described conditions have been optimised from other known RT/cDNA synthesis protocols. It is generally known that other conditions can be established for optimisation of a particular reverse transcriptase reaction on the basis of protocols well known to one of ordinary skill in the art.

As used herein, the term “primer pair” refers to two primers, one having a forward designation and the other having a reverse designation relative to their respective orientations on a double-stranded DNA molecule which consists of a sense and antisense sequence, such that under the amplification conditions described herein, the forward primer anneals to and primes amplification of the sense sequence and the reverse primer anneals to and primes amplification of the antisense sequence. Primers can be selected for use in the amplification reaction on the basis of, having minimal complementarity with other primers in the reaction (to minimize the formation of primer dimers) and having Tm values with the range of reaction temperatures appropriate for the amplification method, preferably PCR. In addition, primers can be selected to anneal with specific regions of the RNA template such that the resulting DNA amplification product ranges in size from 100 to 500 base pairs in length and most preferably around 300 base pairs in length.

For example, in the conditions described above, the primer pair can consist of the oligonucleotide of SEQ ID NO: 13 as the forward primer and the oligonucleotide of SEQ ID NO: 14 as the reverse primer.

As used herein, the terms “detecting” or “detection” of the amplified DNA refers to qualitatively or quantitatively determining the presence of the amplified DNA strand, which is only synthesized if reverse transcriptase is resistant to the reverse transcriptase inhibitor drug added to the assay mixture. The amplification of the synthesized DNA can be detected by any method for the detection of DNA known in the art. For example, detection of the amplified DNA can be by Southern blot hybridisation assay, by visualization of DNA amplification products of specific molecular weight on ethidium bromide stained agarose gels, by measurement of the incorporation of radiolabeled nucleotides into the synthesized DNA strand by autoradiography or scintillation measurement.

The preferred detection method is by agarose gel electrophoresis using ethidium bromide staining and visualisation under UV light.

The principles of PCR and the conditions for amplification and detection of target nucleic acids are well known in the art and may be found in numerous references known to the skilled artisan, including, for example, U.S. Pat. No. 4,683,195; U.S. Pat. No. 4,683,202 and U.S. Pat. No. 4,965,188; all to Mullis et al. Briefly, a sample suspected of containing a target nucleic acid is heated to denature double-stranded nucleic acid in the presence of two oligonucleotide primers that are complementary to target sequences flanking the region to be amplified. The primers anneal to the separated target strands and are extended from each 3′ hydroxyl end by a polymerising agent such as a thermostable polymerase. Double-stranded or single-stranded DNA can be amplified by PCR. RNA can also serve as a target by reverse transcribing RNA into cDNA.

The steps of denaturation, primer annealing and DNA synthesis are carried out at discrete temperatures, and repeated cycles result in exponential accumulation of the target nucleic acid. The PCR vessel is generally a stoppered plastic vessel or a cuvette or pouch as described in U.S. Pat. No. 5,229,297. Reagents for PCR amplification are typically mixed in a single vessel, and generally include primers, nucleoside triphosphates (generally dATP, dCTP, dGTP and dTTP or dUTP), thermostable DNA polymerase, magnesium containing buffer, and target nucleic acid. Reagents and conditions for PCR are well-known to one of ordinary skill in the art, and can be found, for example, in Guatelli et al. (1989) Clin. Microbiol. Rev. 2:217. For amplification of RNA targets, a reverse transcriptase may be utilized in addition to or in lieu of the thermostable DNA polymerase. Thermostable reverse transcriptase are particularly useful, as are thermostable DNA polymerases having reverse transcriptase activity. Methods for PCR amplification of RNA targets are known to one of ordinary skill in the art and described, for example, in U.S. Pat. Nos. 5,176,995, 5,310,652 and 5,322,770.

Detection of the DNA amplified by the PCR is generally conducted in a manner that the DNA is subjected to electrophoresis using agarose gel, acryl amide gel or the like, and then it is subjected to dyeing with nucleic acid-specific dyeing reagents. In the case of detecting double-stranded DNA, usually a fluorescent reagent such as ethidium bromide is allowed to enter between two strands of the DNA and then the fluorescent reagent is excited by an ultraviolet light source. As ethidium bromide entered between the two strands of the DNA emits fluorescence, detection is performed by means of capturing the fluorescence with a CCD camera or the like.

The object of the present invention is accomplished by the amplification, by PCR, of seven specific marker genes or sub-sets thereof and latter separation by electrophoresis of the products of this amplification, followed by appropriate colouring techniques that permit an adequate visualisation of the DNA in the gel including, but not limited to: colouring by silver salts, radioisotopes and enzymes combined with substrates that permit their detection.

It is an object of certain embodiments of the present invention to provide a method of conducting nucleic acid amplification reactions in a single reaction chamber whereby internal primer pairs hybridise to opposing reigns of said target genetic marker genes, and amplification occurs by polymerase chain reaction.

Internal control primer pairs designed to hybridise to opposing strands of a suitable control housekeeping gene such that semi-quantitative comparisons can be made. Such a preferred housekeeping gene can be actin, GADPH, or elongation factors. By measuring the intensity of the internal control signal and comparing that to the signals given by the said genetic marker genes, one can determine the degree of change of expression of said genetic marker gene from the normal levels of expression (i.e. those levels in which no disease state is present).

In the method of the present invention, PCR amplification is accomplished by pre-incubating all PCR reagents and a sample containing a target nucleic acid in the presence of appropriate gene specific primers and a thermostable polymerase enzyme. The resulting reaction mixture is cyclically heated under conditions allowing for the formation and amplification of primer extension products.

The reagents required for PCR are known to persons skilled in the art, and generally include at least two oligonucleotide primers that are sufficiently complementary to conserved regions of the target nucleic acid to hybridise thereto, four different nucleoside triphosphates, a thermostable polymerisation agent and any requisite cofactors for the polymerisation agent. Preferred nucleoside triphosphates are the deoxyribonucleoside triphosphates dATP, dCTP, dGTP and dTTP or dUTP, collectively termed dNTPs. Nucleoside triphosphates are commercially available.

Primers include naturally occurring or synthetically produced oligonucleotides capable of annealing to the target nucleic acid and acting as the point of initiation of nucleic acid synthesis under appropriate conditions, i.e., in the presence of nucleoside triphosphates, a polymerisation agent, suitable temperature, pH and buffer. The primers have sequences sufficiently complementary to the target nucleic acid to hybridise thereto, and are of sufficient length, typically from 10-60 nucleotides, to prime the synthesis of extension products in the presence of a polymerisation agent. Primers may be produced synthetically by automated synthesis by methods well known to one of ordinary skill in the art.

Design considerations for primers are well known in the art. Primers are selected to be substantially complementary to the sequences of the strands of the specific nucleic acid to be amplified, such that the extension product synthesized from one primer, when separated from its complement, can serve as a template for the extension product of the other primer. Preferably, the primers are exactly complementary with the target region. It is underlined that the primer pairs given in the present specification, examples and claims can be replaced by functionally equivalent primers, exhibiting specificity to the marker genes, without departing from the scope of the invention.

The inventors have unexpectedly identified seven (7) marker genes whose specific changes in expression status, collectively or in sub-sets, is indicative of the inflammatory bowel disease condition, UC. This opens the possibility of a rapid detection protocol at the molecular level designed to aid the examining physician in correctly predicating disease type.

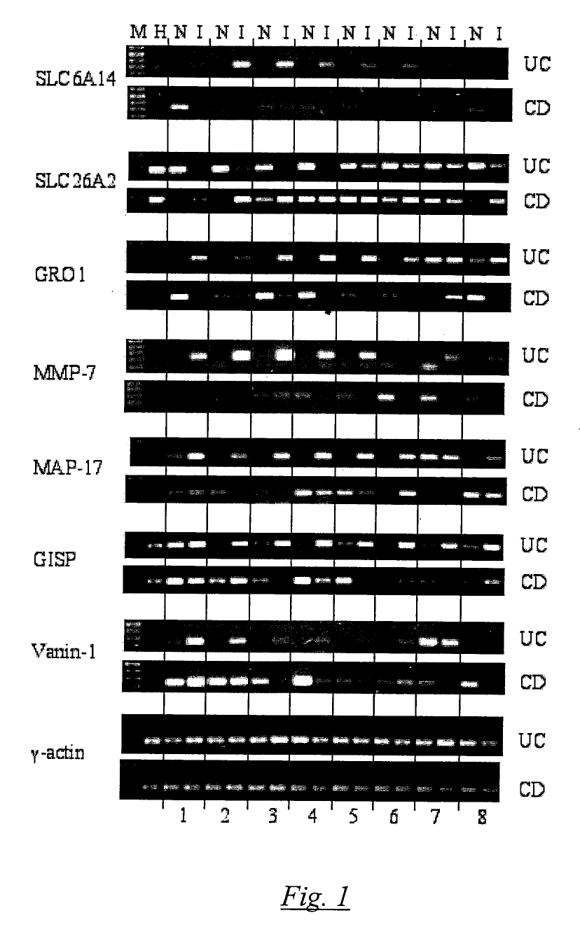

The expression profile in inflamed and non-inflamed tissue is exemplified in FIG. 1 and in Table 1 below.

TABLE 1
Expression profiles in inflamed and non-inflamed tissue

| | Inflamed tissue | | Non-inflamed tissue | |
|---|---|---|---|---|
| | Ulcerative | | Ulcerative | |
| Marker Gene | colitis | Crohn's disease | colitis | Crohn's disease |
| SLC6A14 | X | — | — | X |
| SLC26A2 | — | X | XX | X |
| GRO1 | X | — | — | X |
| MMP-7 | XX | — | X | — |
| MAP-17 | X | — | — | X |
| GISP | XX | X | X | XX |
| Vanin-1 | X | (X) | — | X |
| Gamma actin | X | X | X | X |
| (control) | | | | |

X, and XX indicates the degree of expression;
(X) indicates a result which is likely to be disregarded in light of the overall picture;
— denotes lack of expression

The genetic markers are solute carrier family 6 member 14 (SLC6A14), solute carrier family 26 member 2 (SLC26A2), CXC chemokine growth-related oncogene-alpha (Gro-alpha) or (CXCL-1), Matrilysin also known as matrix metalloproteinase-7 (MMP-7), gastro-intestinal secretory protein (GISP) also know as regenerating gene type IV (Reg IV), membrane associated protein 17 (MAP-17), and vanin-1. See Table 2.

TABLE 2
Target genetic marker genes

SEQ ID NO: 1 (GENBANK Acc No NM_007231)
SEQ ID NO: 2 (GENBANK Acc No NM_000112)
SEQ ID NO: 3 (GENBANK Acc No NM_001511)
SEQ ID NO: 4 (GENBANK Acc No BC003635)
SEQ ID NO: 5 (GENBANK Acc No NM_005764)
SEQ ID NO: 6 (GENBANK Acc No BC017089)
SEQ ID NO: 7 (GENBANK Acc No NM_004666)

Polymerisation agents are compounds that function to accomplish the synthesis of the primer extension products. The polymerisation agents are thermostable, i.e., not permanently inactivated when heated for brief periods to temperatures typically used in PCR for denaturation of DNA strands, e.g., 93-95° C., and are preferentially active at high temperatures. In a preferred embodiment the polymerisation agent is a thermostable DNA polymerase, including, for example, DNA polymerase obtained from thermophilic bacteria such as, Thermococcus litoralis, Bacillus stearothermophilus, Methanothermus fervidus, Thermus aquaticus, T. filiformis, T. flavus, T. lacteus, T. rubens, T. ruber and T. thermophilus; or from thermophilic archaebacteria such as Desulfurococcus mobilis, Methanobacterium thermoautotrophilcum, Sulfolobus solfataricus, S. acidocaldarius and Thermoplasma acidophilum. In a most preferred embodiment, the polymerisation agent is Thermus aquaticus (Tat) polymerase, T. thermophilus (Tth) polymerase or Thermococcus litoralis polymerase. Thermostable reverse transcriptase and DNA polymerases having reverse transcriptase activity are also contemplated as polymerisation agents.

The thermostable polymerases may be obtained commercially or by methods known in the art. In particular, Taq polymerase is available commercially in recombinant and native form (Perkin Elmer-Cetus) or can be produced by the method described by Lawyer et al., (1989) or in U.S. Pat. No. 4,889,818. Tth polymerase is commercially available from Finnzyme Co., Finland and from Toyobo Co., Japan. Thermococcus litoralis polymerase is commercially available from New England Biolabs and can be produced by the method described in U.S. Pat. No. 5,322,785.

Antibodies specific for the thermostable polymerisation agents may be included in the pre-amplification step to inhibit the polymerisation agent prior to amplification. Antibodies can be produced by methods known to one of ordinary skill in the art and found, for example, in Harlowe et al. (1988) Antibodies: A Laboratory Manual, Cold Spring Harbor, N.Y. In accordance with the present invention, the term antibodies includes monoclonal and polyclonal antibodies produced by conventional methodologies, recombinantly produced antibodies, and chemically or recombinantly produced fragments of antibodies, such as Fab fragments. In a preferred embodiment, the antibodies are monoclonal.

In a preferred embodiment of the present invention, the antibody is a monoclonal antibody against Taq polymerase, Tth polymerase, or Thermococcus litoralis polymerase. In a more preferred embodiment, the antibody is a monoclonal antibody against Taq polymerase. Monoclonal antibodies against Taq polymerase are known in the art and described, for example, in U.S. Pat. No. 5,338,671. In accordance with the present invention, antibodies defined as specific for polymerisation agent are those antibodies that are capable of inhibiting the enzymatic activity of the polymerisation agent at temperatures from about 20-40° C. The antibodies of the invention are inactivated by elevated temperatures used during PCR thermal cycling. The ability of the antibodies to inhibit enzymatic activity of the polymerase can be determined by assays known to one of ordinary skill in the art, as described, for example, by Sharkey et al. (1994).

The present invention provides a method for the amplification of a target nucleic acid, and optionally, the subsequent detection of the nucleic acid, in a sample suspected of containing the target nucleic acid. The sample may be any sample suspected of containing a target nucleic acid, including, for example, a tissue sample, blood, hair, body fluid, bacteria, virus, fungus, bacterial infected cell, virally infected cell, and so on. The target nucleic acid may be DNA or RNA. A sufficient number of bases at both ends of the sequence to be amplified must be known in order to design primers capable of hybridising to the different strands of the target nucleic acid at suitable positions for PCR amplification. The target nucleic acid may be extracted or partially extracted from the tissue sample prior to PCR, for example, by removing proteins or cellular material from the sample. Methods for extracting nucleic acids from samples are known to one of ordinary skill in the art and may be found, for example, in Sambrook et al., (1989) and Saiki et al., (1985).

In a preferred embodiment, biopsies resected from the gastro-intestinal tract and from area believed to be exhibiting signs of the disease is particularly preferred as the source of material.

In the method of amplification of the present invention, the sample or a preparation of nucleic acids extracted from the sample is contacted with the reagents typically used for PCR, including at least two oligonucleotide primers modified to contain at least one phosphorothioate linkage, four different nucleoside triphosphates, a thermostable polymerisation agent, and an appropriate buffer, and further with an exonuclease to form a reaction admixture. In another embodiment, an antibody specific for the polymerisation agent is included in the admixture.

The conventional PCR reagents, including primers, nucleoside triphosphates, polymerisation agent, and appropriate buffer are utilized at concentrations generally appropriate for PCR and known to one of ordinary-skill in the art. In a preferred embodiment, the nucleoside triphosphates are dATP, dCTP, dGTP and dTTP. In a preferred embodiment the polymerisation agent is a thermostable DNA polymerase. Preferred DNA polymerases are Taq polymerase, Tth polymerase and Thermococcus litoralis polymerase. Taq polymerase is particularly preferred.

The amplification method is preferably conducted in a continuous, automated manner. Appropriate instrumentation for automated PCR is well-known to the ordinarily skilled artisan and described, for example, in U.S. Pat. Nos. 4,965,188, 5,089,233 and 5,229,297. The skilled artisan can also easily detect amplified product, for example, by separating PCR products by agarose gel electrophoresis and visualizing by ethidium bromide staining, or detecting by hybridisation with a labeled probe capable of hybridising with the amplified nucleic acid or a variety of other detection methods well-known to one of ordinary skill in the art.

One embodiment of the invention is thus a method for the differentiation between ulcerative colitis and Crohn's disease based on the analysis of gene expression profiles in biopsy samples obtained from inflamed and non-inflamed areas in the intestines of a patient, wherein the expression levels of at least two of a number of marker genes are determined, said at least two marker genes chosen among SEQ ID NO:1, SEQ ID NO:2, SEQ ID NO:3, SEQ ID NO:4, SEQ ID NO:5, SEQ ID NO:6, and SEQ ID NO:7.

Another embodiment of the invention is a method wherein the expression levels of SEQ ID NO:1 and SEQ ID NO:2 are determined and wherein the expression of SEQ ID NO:1 in inflamed tissue and lack of expression in non-inflamed tissue, together with the lack of expression of SEQ ID NO:2 in inflamed tissue is taken as an indication of ulcerative colitis.

A third embodiment of the invention is a method wherein the expression levels of SEQ ID NO:1, SEQ ID NO:2, and, SEQ ID NO:3 are determined, and wherein the expression of SEQ ID NO:1 in inflamed tissue and lack of expression in non-inflamed tissue, together with the lack of expression of SEQ ID NO:2 in inflamed tissue, and the expression of SEQ ID NO:3 in inflamed tissue and lack of expression in non-inflamed tissue, is taken as an indication of ulcerative colitis.

A fourth embodiment is a method wherein the expression levels of SEQ ID NO:1, SEQ ID NO:2, and SEQ ID NO:4 are determined, and wherein the expression of SEQ ID NO:1 in inflamed tissue and lack of expression in non-inflamed tissue, together with the lack of expression of SEQ ID NO:2 in inflamed tissue, and the expression of SEQ ID NO:4 in inflamed tissue and lack of expression in non-inflamed tissue, is taken as an indication of ulcerative colitis.

A fifth embodiment is a method wherein the expression levels of SEQ ID NOs:1-7 are determined, and wherein the expression of SEQ ID NO:1, SEQ ID NO:3, SEQ ID NO:4, SEQ ID NO:5, SEQ ID NO:6, and SEQ ID NO:7 in inflamed tissue and lack of expression in non-inflamed tissue, together with the lack of expression of SEQ ID NO:2 in inflamed tissue, is taken as an indication of ulcerative colitis.

According to a preferred embodiment, the method of any one of the above embodiments includes a step wherein the expression level of each marker gene is determined through nucleic acid amplification of said genes using gene specific primers, and determination of the amplification results. The nucleic acid amplification is preferably performed using PCR and the gene specific primers preferably chosen among SEQ ID NOs:13-26.

The determination of the amplification results is preferably performed using ethidium bromide staining and visualisation under UV light.

The present invention further provides a kit for PCR comprising, in the same or separate containers, a thermostable polymerisation agent, and primer pairs designed to allow PCR amplification of said target genes. Additional containers can also be provided for the inclusion of, for example, additional antibodies specific to the PCR polymerisation agent and reagents for PCR, including, for example, nucleoside triphosphates, primers and buffers.

Consequently, the present invention makes available a kit for the differentiation between ulcerative colitis and Crohn's disease based on the analysis of gene expression profiles in biopsy samples obtained from inflamed and non-inflamed areas in the intestines of a patient, said kit including gene specific primer pairs directed to at least two marker genes chosen among SEQ ID NO:1, SEQ ID NO:2, SEQ ID NO:3, SEQ ID NO:4, SEQ ID NO:5, SEQ ID NO:6, and SEQ ID NO:7.

Said gene specific primer pairs are preferably chosen among SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; and SEQ ID NO:25 and SEQ ID NO:26.

According to one embodiment of the invention, said specific primers are SEQ ID NO:13 and SEQ ID NO:14; SEQ ID NO:15 and SEQ ID NO:16; and SEQ ID NO:17 and SEQ ID NO:18.

According to another embodiment of the invention, said specific primers are SEQ ID NO:13 and SEQ ID NO:14; SEQ ID NO:15 and SEQ ID NO:16; and SEQ ID NO:19 and SEQ ID NO:20.

The kit according to the invention further preferably comprises a thermostable polymerisation agent and requisite cofactor(-s). In a preferred embodiment the polymerisation agent is a DNA polymerase. In a more preferred embodiment the polymerase is Taq polymerase, Tth polymerase, or Thermococcus litoralis polymerase. Taq polymerase is particularly preferred. The preferred antibody is a monoclonal antibody specific for Taq polymerase.

The oligonucleotide primers preferably are 19-25 nucleotides in length and are designed as primer-pairs that will under standard conditions anneal to the target DNA of said genetic marker genes. In this case seven primer pairs are provided that will allow successful amplification of the seven said genetic marker genes. The specific primers used are presented in Table 3.

TABLE 3
Primer-pairs

| | | |
|---|---|---|
| SLC6A14 (for) | 5′-GTG CTG AGA TTA CAG GTG TGA GCC-3′ | (SEQ ID NO: 13) |
| SLC6A14 (rev) | 5′-CCC TTC ACA CCT CCC CCA ATT AGA-3′ | (SEQ ID NO: 14) |
| SLC26A2 (for) | 5′-GTG GAG AGA GGG AAA GAA TGT TGC-3′ | (SEQ ID NO: 15) |
| SLC26A2 (rev) | 5′-CCA GTT TAG GAC AGA TTC CAT GGG-3′ | (SEQ ID NO: 16) |
| GRO1 (for) | 5′-GTG CCT AAT GTG TTT GAG CAT CGC-3′ | (SEQ ID NO: 17) |
| GRO1 (rev) | 5′-GCC CCT TTG TTC TAA GCC AGA AAC-3′ | (SEQ ID NO: 18) |
| MMP7 (for) | 5′-CAG GCA GAA CAT CCA TTC ATT CAT TC-3′ | (SEQ ID NO: 19) |
| MMP7 (rev) | 5′-GAC ATC TAC CCA CTG CAA GTA TAG-3′ | (SEQ ID NO: 20) |
| MAP17 (for) | 5′-CCG TCG GAA ACA AGG CAG ATG GAG-3′ | (SEQ ID NO: 21) |
| MAP17 (rev) | 5′-GAA GGA CGT GTG AGC AGG ATG GGA-3′ | (SEQ ID NO: 22) |
| GISP (for) | 5′-GGT GGG AAC AAG CAC TGT GCT GAG-3′ | (SEQ ID NO: 23) |
| GISP (rev) | 5′-GGC TGG AGA TGC ACT CTT CTA GAC-3′ | (SEQ ID NO: 24) |
| Vanin1 (for) | 5′-GCC AGC AAA ACA TCA TTT TGA GAC-3′ | (SEQ ID NO: 25) |
| Vanin1 (rev) | 5′-GCC TAT CAC CAA CAC ATC AAT ATG-3′ | (SEQ ID NO: 26) |

The genetic markers disclosed are, solute carrier family 6 member 14 (SLC6A14), as given by SEQ ID NO:1, solute carrier family 26 member 2 (SLC26A2) as given by SEQ ID NO:2, CXC chemokine growth-related oncogene-alpha (Gro-alpha) or (CXCL-1) as given by SEQ ID NO:3, Matrilysin also known as matrix metalloproteinase-7 (MMP-7) as given by SEQ ID NO:4, gastro-intestinal secretory protein (GISP) also know as regenerating gene type IV (Reg IV) as given by SEQ ID NO:5, membrane associated protein 17 (MAP-17) as given by SEQ ID NO:6, and Vanin-1 as given by SEQ ID NO:7. See Table 2, supra.

In the methods illustrated by the examples, a number of method based sequences were used. These are presented in Table 4.

TABLE 4
Method based sequences

| | |
|---|---|
| (SEQ ID NO: 8) | 5′-TAG TCT ATG ATC GTC GAC GGC TGA TGA AGC |
| | GGC CGC TGG AGT TTT TTT TTT TTT TTT TTV-3′ |
| (SEQ ID NO: 9) | 5′-TGA TGA AGC GGC CGC TGG-3′ |
| (SEQ ID NO: 10) | 5′-TTC ATC AGC CGT CGA CGA TC-3′ |
| (SEQ ID NO: 11) | 5′-CGT AAG CTT GGA TCC TCT AGA GC-3′ |
| (SEQ ID NO: 12) | 5′-TGC AGG TAC CGG TCC GGA ATT CC-3′ |

Solute carrier (SLC) proteins comprise of a very large family of energy dependent transport molecules and have critical physiological roles in nutrient transport and may be utilized as a mechanism to increase drug absorption. However, there is limited understanding of these proteins at the molecular level due to the absence of high-resolution crystal structures.

In total, 1-2% of adults and 6-8% of children suffering from kidney stones have cystinuria, a defect in the transport of amino acids, which leads to high concentrations of cystine in the urine. Two genes have been implicated, solute carrier family 3 (cystine, basic and) neutral amino acid transporter, member 1 (SLC3A1) coding for the protein related to the system of amino-acid transporter, and solute carrier family 7, member 9 (SLC7A9). Both of these solute carriers are believed to be involved in stone formation which may ultimately lead to urinary tract infection and, eventually, renal failure.

The inventors have identified two known solute carriers (SLC6A14 and SLC26A2) whose expression is significantly altered in IBD. To the best knowledge of the inventors, this is the first reporting of the potential involvement of solute carriers in inflammatory bowel diseases. It is therefore a novel finding that solute carriers might contribute to the pathogenesis of IBD.

CXC chemokine growth-related oncogene-alpha (Gro-alpha also known as GRO1) is as described a cytokine and as such can alter the migratory responses of numerous cell types in local areas of inflammation. It and has been described to be over expressed in human inflamed corneas (Spandau et al., 2003) and in addition, it has also been shown that rats chemically induced to exhibit inflammation of the gut show up-regulated levels of GRO1 (Hirata et al., 2001). Using a cDNA microarray approach, Heller et al., 1997 describes novel participation of the, chemokine Gro alpha in rheumatoid arthritis and inflammatory bowel disease, however the invention presented here describes, in the inventors best knowledge for the first time, that GRO1 while over expressed in conditions of UC is down regulated in conditions of CD. While it is described in Isaacs et al, 1992, that expression of GRO1 in UC is higher than that seen in CD, here it has been demonstrated that there exists an inverse correlation of UC verses CD with respect to GRO1 expression levels. Lastly Lawrence et al., 2001 describes identifying GRO1 as being up-regulated in UC, but the design of the study was such that biopsy samples where pooled before analysis, therefore is was not possible to know whether GRO1 was up-regulated in more than 1 patient.

Matrilysin or (matrix metalloproteinase-7) was first discovered in the involuting rat uterus; it has also been known as uterine metalloproteinase, putative metalloproteinase (Pump-1), and matrix metalloproteinase 7 (MMP-7). It is the smallest member (28 kDa) of a family of 15 MMPs that together are able to degrade most of the macromolecules of the extra cellular matrix. This family is briefly reviewed; all members are zinc metalloproteinases that occur in zymogene form with the active site zinc blocked by cysteine. Matrilysin can degrade a wide range of gelatins, proteoglycans, and glycoproteins of the matrix and can activate several other MMPs including collagenase (reviewed in Woessner, 1996).

It is frequently expressed in various types of cancer including colon, stomach, prostate, and brain cancers. Previous studies have suggested that matrilysin plays important roles in the progression and metastasis of colon cancer. Recently it has been described by Newell et al., 2002 that there is an increase of matrilysin expression at different stages of UC-associated neoplasia. This work however does not determine whether such increased expression is a result of UC or rather due to the presence of neoplasia.

Membrane associated protein 17 (MAP-17) or otherwise know as DD96, is a small protein has to date no described function. Regarding GISP, again there is little known. In both cases neither has been described as potentially involved in inflammation.

Pantetheinase (EC 3.5.1.) is an ubiquitous enzyme which in vitro has been shown to recycle pantothenic acid (vitamin B5) and to produce cysteamine, a potent anti-oxidant. The enzyme is encoded by the Vanin-1 gene and is widely expressed in mouse tissues. Vanin-1 is a GPI-anchored pantetheinase, and consequently an ectoenzyme. It has been suggested that Vanin/pantetheinase might be involved in the regulation of some immune functions maybe in the context of the response to oxidative stress (Pitari et al., 2000).

To the best knowledge of the inventors, this is the first description of the potential role of Vanin-1 in IBD.

While the present invention has been described with specificity in accordance with certain of its preferred embodiments, the following examples are only intended to illustrate the invention and not to limit the same. While they are typical of methods and method steps that might be used, others known to those skilled in the art may be adopted without resorting to undue experimentation.

### EXAMPLES

### Example 1

### Collection of Biopsy Material

The biopsies were taken from patients who were selected on the basis of clinical and pathological evidence of having the inflammatory condition of CD or UC. A total of three biopsies were collected from an inflamed site in the colon, together with three biopsy samples from a non-inflamed region of a single individual patient. This was done for a total of 16 different patients of which eight were diagnosed for CD (patient 1-8) and eight for UC (patient 9-16). The UC patient group comprised 2 females and 6 males, the age range being 29-77 years. The CD age group correspondingly 3 females and 5 males, age range 27-59.

The biopsies from each anatomical site of one patient were pooled and total RNA isolated using Qiagen RNEASY Kit, RNA isolation kit, and a Pellet Pestel Motor Homogenizer according to the manufacturer's protocol. In this way 32 samples of total RNA were isolated, two samples per patient: inflamed (target) and non-inflamed (control).

### Example 2

### Performing cDNA Synthesis of the RNA

Two microgram of each RNA sample (32 in total) was used for a first strand cDNA synthesis using 10 μM of the Oligo-dT-primer dT-joint (5′-TAG TCT ATG ATC GTC GAC GGC TGA TGA AGC GGC CGC TGG AGT TTT TTT TTT TTT TTT TTV-3′ (SEQ ID NO:8) introducing to every synthesised cDNA molecule three restriction enzyme cutting sites: SalI, NotI and BpmI. The buffer, desoxynucleotide triphosphates (dATP, dCTP, dGTP and dTTP) and the enzyme reverse transcriptase (SUPERSCRIPT II) were taken from Gibco BRL and the reactions were performed according to the manufacturer's guidelines. The reaction mixture for first strand synthesis excluding the enzyme was pre-incubated for 5 min at 65° C. in a PCR machine (PCR sprint from Hybaid), chilled on ice, and then preheated to 42° C., before the enzyme SUPERSCRIPT II, reverse transcriptase, was added and incubated for 1 h at 42° C. in a PCR machine (PCR sprint from Hybaid).

For the second strand synthesis, 41 μl second strand buffer mix were added to the reactions according to the provided protocol (Gibco BRL) and 4 μl E. coli Polymerase I (New England Biolabs), 1.5 μl E. coli DNA ligase (New England Biolabs) and 0.7 μl Rnase H (Gibco BRL) in a total volume of 160 μl. The reactions were incubated for 2.5 h at 16° C. in the PCR machine PCRsprint and then purified using the Qiagen PCR Purification Kit according to the protocol provided. Every sample (32 in total) was eluted with 32 μl of elution buffer and 26 μl of each sample was used for the following steps.

### Example 3

### Amplification of the 3′-Termini of the cDNAS

Due to limited amounts of material obtained from such biopsies, a pre-amplification step was necessary. For in vitro amplification of the 3′-end of cDNAs, 26 μl of cDNA from every sample was digested with 10 U of the restriction enzyme DpnII in a volume of 30 μl for 3 h at 37° C. The cut cDNAs were purified once more using Qiagen PCR purification Kit and the cDNAs were eluted in 47 μl elution buffer. The following circular ligation step was performed in a volume of 50 μl including 44 μl of the DpnII cut cDNA and 2000 U T4 DNA ligase (New England Biolabs). These reaction mixtures were incubated at 22° C. for 1 h, heat inactivated by 65° C. for 10 min and 25 μl of each reaction mixture was used for the amplification step. A mixture for 5 reactions per sample was put together (5×50 μl=250 μl in total) containing 25 μl cDNA (DpnII cut and circular ligated), 25 μl 10× Advantage 2 PCR buffer (Clontech), 5 μl joint-Not primer (10 μmol/μl; 5′-TGA TGA AGC GGC CGC TGG-3′ (SEQ ID NO:9)), 5 μl joint-Sal primer (10 pmol/μl; 5′-TTC ATC AGC COT CGA CGA TC-3′ (SEQ ID NO:10), 5 μl 10 mM dNTP mix and 5 μl 50× Advantage 2 Taq-Polymerase (Clontech). For each sample the mix was distributed into 5 PCR reaction tubes and PCR performed under the following conditions: 1 min 94° C. then 16× (20 sec 94° C., 20 sec 55° C., 1 min 72° C.).

Four of the reactions per sample were removed and placed on ice and the optimal cycle number was determined with one of the reactions per sample. The optimal cycle number was determined to 18 cycles for all 32 samples, thus for the remaining four reactions per sample two additional cycles [2× (20 sec 94° C., 20 sec 55° C., 1 min 72° C.)] were performed. The 4 PCR reactions per sample were subsequently purified using the Qiagen PCR purification Kit. For the purification, the four reactions per sample were pooled (total of 200 μl) and then eluted with 34 μl elution buffer. The purified reactions were the starting material for the identification of the differentially expressed genes protocol.

### Example 4

### Isolation of the Differentially Expressed cDNA (Subtraction Protocol) from Human Biopsies

Isolation of differentially expressed cDNAs was performed according to the protocol outlined in (von Stein O. D., 2001) with minor modifications to the protocol.

### Example 5

### Screening for the Differentially Expressed Genes

Upon construction of a cDNA library, 2.000 clones were plated out from each subtraction on one 22 cm² agar plate. From these plates 384 colonies were picked and placed in 384 well plates with 70 μl LB medium/well (see Maniatis et al., Molecular cloning laboratory book, Appendix A. 1) (+ampicillin 100 mg/ml) using BioPick machine of BioRobotics (Cambridge, UK). The bacterial clones were incubated over night at 37° C. and then used for colony PCR. This PCR was performed in 384 PCR well plates in a volume of 20 μl per sample. One PCR reaction included: 2 μl 10×PCR buffer, 0.4 μl Sport-Not primer (10 μmol 5′-CGT AAG CTT GGA TCC TCT AGA GC-3′ (SEQ ID NO:11), 0.4 μl of Sport-Sal primer (10 μmol 5′-TGC AGG TAC CGG TCC GGA ATT CC-3′ (SEQ ID NO:12)), 1.6 μl dNTP mix (25 mM each), 0.4 μl 0.1% Bromphenol blue and 0.5 μl DynAzyme Taq-polymerase (2 U/μl; Finnzyme). A master mix for all reactions was prepared, distributed and then inoculated with a 384 plastic replica. The PCR cycling parameters were: 2 min 94° C., 37 times (30 sec 94° C.; 30 sec 50° C., 1 min 72° C.) and 5 min 72° C.

Following amplification, PCR reactions were spotted on HYBOND N+ membrane (Amersham) using Microgrid TAS of BioRobotics. All clones were spotted in duplicate and genomic DNA was used as guide dots. On one filter 384 genes of all four subtractions were positioned. 24 duplicates were made for analyses by hybridisation with different radioactive cDNA probes.

These filters were then hybridised with the radioactive labeled subtracted cDNAs of all eight patients. Sixteen filters were used in 16 different hybridisation experiments. For that 1 μl of the cDNAs were used for the labelling with Klenow polymerase. The hybridisation protocol was that of Church-protocol as outlined in (Maxam and Gilbert 1984).

Phospho-imager Fuji film BAS 1800II with BAS 1800 III R program and ARRAYVISION version 6.0 (Imaging Research Inc) were used to determine the degree of differentially expression. Genes which were differentially expressed in at least three of the eight patients with an induction or reduction rate of three fold were sequenced and BLAST analysis performed to identify these isolated differentially expressed genes.

### Example 6

### Confirmation of True Differential Expression

Several genes showed strong dysregulation during these analyses. To confirm these data RT-PCR were performed using gene-specific oligonucleotides and un-amplified cDNA material derived from the same eight patients. Approximately, 21 μg total RNA of inflamed and non-inflamed tissue (same as used for the subtraction) were taken for a first strand cDNA synthesis as described in example 5. After the cDNA synthesis the samples were incubated for 3 min at 96° C. and then 1:10 diluted with distilled water.

10 μl of further 1:10 dilutions were taken for one 50 μl PCR reaction. The PCR reaction included: 5 μl 10×PCR buffer, 1 μl forward primer (10 μmol/μl) and 1 μl reverse primer (10 μmol/μl) of the specific genes (SEQ ID NOs: 1-7)), 0.5 μl dNTP mix (25 mM each) and 0.5 μl DynAzyme Taq-polymerase (2 U/μl; Finnzyme). A master mix minus the cDNA for the reactions was prepared, distributed and then the cDNA added. The PCR cycling parameters were: 1 min 94° C., 26-35 times (30 sec 94° C.; 30 sec 55° C., 1 min 72 C) and 5 min 72° C. The cycle number was dependent which gene fragments were amplified. The primer pairs were those shown in Table 3.

These analyses lead to the identification of seven (7) genetic markers whose change in expression status, collectively or in sub-sets, when compared to normal tissue would allow for a correct predication rate of over 90% with regard to UC. To confirm these findings, the markers where further screened against a larger sample collection of biopsies.

### Example 7

### High Throughput Screening

To confirm the preliminary results of the RT-PCR using the cDNA of eight UC patients and three CD patients (see Example 6), it was decided to analyse the expression of all isolated genes in a large scale. For that purpose, all genes that could be isolated from the screening were spotted on a Hybond N+ membrane (Amersham) using the Microgrid TAS (BioRobotics). As described in Example 5 the genes were amplified via a colony PCR for the spotting.

This master filter membrane was then synthesized 240 times and hybridised with radioactive labelled cDNA deriving from biopsies from 50 individual UC patients and 50 individual CD patients. The biopsies were taken from the inflamed and non-inflamed area of the patients in the left side of the colon. As a baseline control the biopsies deriving from the left side of the colon of five healthy people were pooled.

### Example 8

### Verification through Blind Study

To provide greater statistical weight, it was necessary to perform expression analyse of the seven genetic markers on “blind” biopsy samples, whereby it was not known whether the biopsy was derived from a patient suffering from UC or CD. As described previously, RT-PCR analysis of the said genetic markers was performed and by combining the total picture of expression patterns resulting from said genetic markers it was possible to determine with over 90% certainty the correct form of IBD.

Analysis of the results (Blind study) show that already the combination of SEQ ID NOs:1 and 2 give a reliable result, whereas the combination of SEQ ID NOs:1, 2 and 3 or the combination of SEQ ID NOs:1, 2, and 4 give a further improved result. The preliminary results indicate that an accuracy of about 90% was reached using combination of SEQ ID NOs:1, 2, 3 and 4. It was shown that the use of the complete set of SEQ ID NOs:1 through 7 resulted in an accuracy of more than 90%.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

### REFERENCES

Breuning M H, Hamdy N A (2003). From gene to disease; SLC3A1, SLC7A9 and cystinuria. Ned Tijdschr Geneeskd. 147:245-7.
Guatelli J C, Gingeras T R, Richman D D (1989). Nucleic acid amplification in vitro: detection of sequences with low copy numbers and application to diagnosis of human immunodeficiency virus type 1 infection. Clin Microbiol Rev. 2:217-26.
Harlowe E and Lane D (1989). Antibodies. A laboratory manual, Cold Spring Harbour, N.Y.
Heller R A, Schena M, Chai A, Shalon D, Bedilion T, Gilmore J, Woolley D E, Davis R W (1997). Discovery and analysis of inflammatory disease-related genes using cDNA microarrays. Proc Natl Acad Sci USA. 94:2150-5.
Hirata I, Murano M, Nitta M, Sasaki S, Toshina K, Maemura K, Katsu K (2001). Estimation of mucosal inflammatory mediators in rat DSS-induced colitis. Possible role of PGE(2) in protection against mucosal damage. Digestion. 63 Suppl 1:73-80.
Isaacs K L, Sartor R B, Haskill S (1992). Cytokine messenger RNA profiles in inflammatory bowel disease mucosa detected by polymerase chain reaction amplification. Gastroenterology. 103:1587-95.
Lawrance I C, Fiocchi C, Chakravarti S (2001). Ulcerative colitis and Crohn's disease: distinctive gene expression profiles and novel susceptibility candidate genes. Hum Mol Genet. 10:445-56.
Lawyer, F C, Stoffel, S, Saiki, R K, Myambo K, Drummond R, Gelfand D H (1989). Isolation, characterization, and expression in Escherichia coli of the DNA polymerase gene from Thermus aquaticus. J. Biol. Chem. 2641:6427-37.
Newell, K J, Matrisian, L M, Driman, D K (2002). Matrilysin (matrix metalloproteinase-7) expression in ulcerative colitis-related tumorigenesis. Mol. Carcinog. 34:59-63.
Pitari, G, Malergue F, Martin F, Philippe J M, Massucci M T, Chabret C, Maras B, Dupre S, Naquet P, Galland (2000). Pantetheinase activity of membrane-bound Vanin-1: lack of free cysteamine in tissues of Vanin-1 deficient mice. FEBS Lett. 483:149-54.
Saiki, R K, Scharf S, Faloona F, Mullis K B, Horn G T, Erlich H A, Arnheim N (1985). Enzymatic amplification of beta-globin genomic sequences and restriction site analysis for diagnosis of sickle cell anemia. 1985. Biotechnology. 1992; 24:476-80.
Sambrook, J, Russell D W, Sambrook J (1989). Molecular cloning, Cold Spring Harbor Press, Cold Spring Harbor, N.Y.
Sharkey, D J, Scalice E R, Christy K G Jr., Atwood S M, Daiss J L (1994). Antibodies as thermolabile switches: high temperature triggering for the polymerase chain reaction. Biotechnology (N Y). 12:506-9.
Spandau, U H, Toksoy A, Verhaart S, Gillitzer R, Kruse F E. High expression of chemokines Gro-alpha (CXCL-1), IL-8 (CXCL-8), and MCP-1 (CCL-2) in inflamed human corneas in vivo. Arch Opthalmol. 2003 June; 121(6):825-31.
von Stein, O. D. Isolation of differentially expressed genes through subtractive suppression hybridization. Methods Mol Biol 2001, 175:263-78.
Woessner, J F Jr. Regulation of matrilysin in the rat uterus. Biochem Cell Biol. 1996; 74(6):777-84.

What is claimed is:

1. A method for the differentiation between ulcerative colitis and Crohn's disease based on the analysis of gene expression level profiles in biopsy samples obtained from inflamed colon tissue of a human patient, comprising determining expression levels of at least two marker genes in a biopsy sample from inflamed colon tissue and in a biopsy sample from non-inflamed colon tissue, from the patient, wherein said at least two marker genes are SEQ ID NO: 3 and SEQ ID NO: 5, and wherein expression of SEQ ID NO: 3 and SEQ ID NO: 5 in inflamed tissue and the lack of expression of SEQ ID NO: 3 and SEQ ID NO: 5 in non-inflamed tissue is indicative of ulcerative colitis.

2. The method according to claim 1, wherein the expression of each marker gene is determined through nucleic acid amplification of said genes using specific primers, and determination of the amplification results.

3. The method according to claim 1, wherein the nucleic acid amplification is performed using PCR and the gene specific primers are SEQ ID NO:17, 18, 21, and 22.

4. The method according to claim 1, wherein the determination of the amplification results is performed using ethidium bromide staining and visualisation under UV light.

5. A method for the differentiation between ulcerative colitis and Crohn's disease based on the analysis of gene expression level profiles in biopsy samples obtained from inflamed colon tissue of a human patient, comprising determining expression levels of at least two marker genes in a biopsy sample from inflamed colon tissue and in a biopsy sample from non-inflamed colon tissue, from the patient, wherein said at least two marker genes are SEQ ID NO: 3 and SEQ ID NO: 5, and wherein expression of SEQ ID NO: 3 and SEQ ID NO: 5 in non-inflamed tissue and the lack of expression of SEQ ID NO: 3 and SEQ ID NO: 5 in inflamed tissue is indicative of Crohn's disease.

6. The method according to claim 5, wherein the expression of each marker gene is determined through nucleic acid amplification of said genes using specific primers, and determination of the amplification results.

7. The method acc according to claim 5, wherein the nucleic acid amplification is performed using PCR and the gene specific primers are SEQ ID NO:17, 18, 21, and 22.

8. The method according to claim 5, wherein the determination of the amplification results is performed using ethidium bromide staining and visualisation under UV light.
